(19) United States
(12) Patent Application Publication
(10) Pub. No.: US 2014/0093843 A1
(43) Pub. Date: Apr. 3, 2014

(54) METHOD AND APPARATUS FOR HARD TISSUE TREATMENT AND MODIFICATION

(71) Applicants: Gregory B. ALTSHULER, Lincoln, MA (US); Andrei V. BELIKOV, St. Petersburg (RU)

(72) Inventors: Gregory B. ALTSHULER, Lincoln, MA (US); Andrei V. BELIKOV, St. Petersburg (RU)

(73) Assignee: RejuveDent LLC, Quincy, MA (US)

(21) Appl. No.: 14/097,703

(22) Filed: Dec. 5, 2013

Related U.S. Application Data
(63) Continuation of application No. 13/571,692, filed on Aug. 10, 2012.
Application No. 13/571,692 is a continuation of application No. 12/488,673, filed on Jun. 22, 2009, now abandoned.
Application No. 12/488,673 is a continuation of application No. PCT/US2007/085676, filed on Nov. 27, 2007.
(60) Provisional application No. 60/867,315, filed on Nov. 27, 2006.

(51) Int. Cl. A61C 13/15; A61K 6/00; A61C 1/00

(52) U.S. Cl. CPC A61C 19/003; A61C 1/0046; A61K 6/0091
USPC 433/215

(57) ABSTRACT

A device and method for forming a texture on a surface of a hard material. Spatial patterns, such as an array of microbeams, are delivered to the tissue through the handpiece. The plurality of microbeams illuminate and ablate the hard material simultaneously. Each of the microbeams has of a sufficient fluence and pulse width to ablate the surface of the hard material and form the texture. Alternatively, one microbeam of a sufficient fluence and pulse width to ablate the surface of the hard material and form the texture is scanned over the surface either manually or in an automatic fashion.

### RELATED APPLICATIONS

This application is Continuation of U.S. application Ser. No. 13/571,692 filed on Aug. 10, 2012, which is a Continuation of U.S. application Ser. No. 12/488,673, filed on Jun. 22, 2009, which is a Continuation of International Application No. PCT/US2007/085676, filed on Nov. 27, 2007, which claims the benefit under 35 USC 119(e) of U.S. Provisional Application No. 60/867,315, filed on Nov. 27, 2006, all of which are incorporated herein by reference in their entirety.

### FIELD OF THE INVENTION

The present invention relates to the field of dental and hard tissue treatment, including, but not limited to, tooth surface hard tissue modification.

### BACKGROUND OF THE INVENTION

Tooth Surface Preparation and Modification

Tooth surface treatment is important for several dental applications:

1. Tooth surface (enamel and dentine) cleaning for prophylactic and esthetic purposes.

2. Tooth surface cleaning and preparation for better adhesion and bonding of filling materials and veneers.

Standard methods of such tooth surface preparation normally include mechanical treatment with a dental handpiece and a prophy cup or chemical etching with a low-pH non-organic acid, such as phosphoric or hydrochloric acids. In the U.S. a patent application Ser. No. 10/596,535 described the use of organic edible acids, such as a citric acid with pH from about 0.5 to about 3 for the same propose. Several studies have demonstrated the effect of tooth surface preparation similar to the acid treatment after sono-abrasion, air abrasion and laser ablation. An adhesive and filing material applied onto the surface of the cavity after the laser ablation had the adhesion strength similar to or better than the adhesion strength after the acid treatment. All of these methods have several limitations, including the low or/and inconsistent adhesion strength of the deposited material. The main reason for that is that the substrate surface properties and the microstructure are inconsistent because of the nature of chemical etching, ultrasound kinetic or traditional laser ablation.

Several methods of tooth surface modification have been developed.

1. Coating the external surface of the tooth or other hard tissues is one of the most effective methods of changing its appearance and protecting it from an acid attack. Several light cured compounds for the protection of the enamel surface, such as BISCOVER™, have been proposed. Such methods are either very destructive (veneers), or cause rapid discoloration and wear, thereby losing their effect (polymer-based coating materials and flowable resin composites).

2. Teeth function in an environment of mechanical, chemical and thermal stress. With normal chewing, a modest stress of 20 MPa is applied to the tooth more than 1000 times a day. Occasional stress can be up to 100 MPa. This cyclic loading occurs in a water-based fluid environment that can have a pH from 0.5 to 8 and the temperature variations of 50° C. Many different restorative materials have been developed, designed to retain their strength and properties in an aggressive environment (for example, ceramic-based porous alumina infiltrated with lanthanum aluminosilicate glass, or porous zirconia later infiltrated with glass). Porcelain, the most popular material, has excellent color properties, but is brittle and relatively easily fractured unless it is reinforced or strengthened. Porcelain restoration treatment also destroys the tooth structure, since it usually requires tooth preparation and is expensive and time consuming. These restorative materials are used for crowns or veneers and, if done properly, provide excellent esthetic appearance and prevent caries. However, the risk of recurrent caries still exists. Since any destruction of the tooth substance is harmful, clinicians have been attempting to develop non-destructive, or minimally destructive methods for tooth restoration. One such area of research involves the use of lasers.

3. Laser modification of the surfaces of teeth or other kinds of hard tissue is a method of selectively heating the superficial layer of the hard tissue to high temperatures below or above the melting temperature of its mineral components. After cooling, a layer of a newly modified material is created on the tooth surface. This layer can be more resistant to an acid attack, have lower porosity, higher hardness and wear resistance than that of the original enamel or dentine. Such selective heating can be achieved in the oral cavity using a laser. The first laser modification of enamel with increased acid resistance was demonstrated in 1964. Subsequently, other lasers have been studied: the UV excimer laser (ArF laser:0.193 μm, the KrF: 248,308 μm), the solid-state laser (Ruby: 0.69 μm, the Nd:YAG 1.06 μm, the Ho:YAG 2.06 μm, the Er:YAG 2.9 μm) and gas lasers (CO₂: 9.6 μm, 10.6 μm). Heating of the enamel up to a temperature between 400-600° C. leads to a significant loss of carbonate and an increase in the enamel's acid resistance. Further heating to the melting temperature (800-1400° C.) of the mineral components of the enamel, but below ablation thresholds, induces a recrystallization process forming a new structure of the superficial layer with better mechanical and acid resistance properties. This effect was demonstrated for the sealing of early pit fissure caries. A 5 min fluoride treatment in carious-like enamel (1.23% acidulated phosphate fluoride gel, pH=4), followed by a laser treatment with a CO₂ laser (9.6 μm wavelength, 1 J/cm2 fluence, 2 μs pulsewidth) dramatically increases the fluoride content in 1 μm of the superficial layer of enamel and significantly increases its acid resistance. Successful tooth surface laser modification requires precise adjustment of the laser parameters. Most studies of the tooth surface laser modification show such side effects as carbonization, tooth darkening, crack formation in the modified enamel layer, and/or instability to thermocycling. In addition, the risk of overheating the tooth pulp exists. Finally, tooth surface laser modification has not been used in daily dental practice and no such product is currently available on the market.

The present invention proposes a new method and apparatus for preparation of dental hard tissue by laser micro ablation (laser microtexturing) of the hard tissue. Such method and apparatus significantly improves adhesion properties of different materials to the hard tissue. After performing the laser micro ablation (laser mico texturing), a regular micro structure is created on the tooth surface and is used for subsequent impregnation the tooth surface with different materials. The tooth surface is then exposed to chemical, photochemical curing or sintering to create a new tooth surface with new esthetic properties and high resistance to caries and wearing.

### SUMMARY OF THE INVENTION

It is the object of the present invention to provide a method of forming a superficial microtextured layer on a surface of a material. The method comprises generating optical radiation in a spectral range from about 100 nm to about 20000 nm; using the optical radiation to form a plurality of microbeams. The next step is to form a spatial pattern of the plurality of the microbeams with or without spatial overlapping between the microbeams and to deliver the spatial pattern to the surface. Then the plurality of the microbeams is used to form a texture in the form of the spatial pattern on the surface of the material by ablating, evaporating, photoetching or modifying the surface and a superficial layer of the material.

It is also an object of the present invention to provide a method of forming a superficial microtextured layer on a surface of a material comprising generating optical radiation in a spectral range from about 100 nm to about 20000 nm, then forming a microbeam and delivering the microbeam to the surface, and scanning the microbeam over the surface to form a texture having an optical pattern by ablating, evaporating, photoetching or modifying the surface and a superficial layer of the material by the microbeam. That method also contemplates scanning the microbeam over the surface manually or by a scanner.

In the inventive methods the material is selected from the group consisting of dental enamel, dentin, dental cementum, composite resin, porcelain, and amalgam. In the embodiment of the method in which one microbeam is generated, forming the texture is comprises sequentially scanning the microbeam over the surface. In the method where a plurality of microbeams is generated, delivering the spatial pattern to the surface comprises projecting a spatial structure of optical radiation onto the surface.

It is also an object of the present invention to provide a device for forming a microtexture on a surface of a hard material comprising a source of optical radiation with a wavelength selected from a range from about 100 nm to about 20000 nm and of a sufficient fluence and pulse width to ablate or modify the surface of the hard material. The device also comprises a handpiece comprising a optical system to form a plurality of microbeams from the optical radiation on the surface of the hard material, each microbeam having a sufficient fluence and pulse width to ablate or modify the surface of the hard material and form the microtexture;

The present invention is also directed to a device for forming microtexture on a surface of a hard material comprising a source of optical radiation with a wavelength selected from a range from about 100 nm to about 20000 nm and of a sufficient fluence and pulse width to ablate or modify the surface of the hard material. The device also comprises a handpiece comprising an optical system forming a microbeam from the optical radiation on the surface of the hard material, the microbeam having a sufficient fluence and pulse width to ablate or modify the surface of the hard material. An optical scanning system guides the microbeam over the surface of the hard material to form the microtexture. In the described device the optical system can be a spatial modulator, which, in turn, can be an array of microlenses, a phase mask, a grating, diffractive optics, or a holographic structure. The spatial modulator can also be a mirror or an array of micromirrors.

The source of optical radiation in the device can be an output of a delivery system or housed in the handpiece. The plurality of microbeams can be a periodic structure or a random pattern. The optical scanning system for guiding the microbeam can serve to guide a continuous wave microbeam.

The inventive device can further comprise synchronizing means coupled with the scanning system for guiding the microbeam synchronously with pluses of the microbeam.

The device can further comprise an array of microlenses as a phase mask disposed in the handpiece between the source of optical radiation and the surface of the hard material.

The optical radiation can be generated by a diode laser, a diode laser or flashlamp pumped solid state laser, or a diode laser pumped fiber laser. The solid state laser or the diode laser pumped fiber laser has an active medium doped by Er or Ho.

It is also the object of the present invention to provide a method of hard tissue modification comprising forming a superficial microtextured layer on the hard tissue; impregnating the superficial microtextured layer with a compound capable of polymerizing when exposed to light; and exposing the compound to light to induce polymerization. Forming the superficial microtextured layer on the hard tissue comprises forming a plurality of microholes or microgrooves having a depth from about 0.5 μm to about 500 μm, a width from about 1 μm to about 250 μm, and a fill factor from about 5% to about 100%. It is contemplated that the plurality of microholes or microgrooves forms a periodic structure.

It is also a method of modification of hard dental material comprising forming a superficial microtextured layer on the hard tissue; impregnating the superficial microtextured layer with particles and forming an impregnated superficial microtextured layer; selectively heating the impregnated superficial microtextured layer to a temperature sufficient to fuse the impregnated superficial microtextured layer; and letting the impregnated superficial microtextured layer to solidify. The particles are organic particles, such as the particles made of polymethylmethacrylate, polycarbide or epoxy.

The particles also can be inorganic particles selected from the group comprising fluoride, germinate, phosphate, lanthanum, zirconium, and silica glasses, porcelain, crystals of quartz, diamond, sapphire, topaz, amethyst, zircon, agate, granite, spinel, fianite, tanzanite, tourmaline crystals selected from the group containing of Ca(NO₃)₂, Ca(OH)₂, BaO₂, CdCl₂, Na₂O—Al₂O₃—SiO₂, Ca(PO₃), CaF₂, Ca₁₀(PO₄)₆(OH)₂, and Ca₁₀(PO₄)₆F₂, and combinations thereof. The method also contemplates selectively heating the the impregnated superficial microtextured layer comprises heating by acoustic energy, electromagnetic energy, comprising light, microwave, radio frequency, and electric current, and combinations thereof.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic illustration of a microstructure of perforated holes.

FIG. 2 is a schematic illustration of a microstructure of perforated holes.

FIG. 3 is a schematic illustration of a microstructure of perforated holes.

FIG. 4 is a schematic illustration of a microstructure of perforated holes.

FIG. 5 is a schematic illustration of holes and a bridge structure.

FIG. 6 is a schematic illustration of one of the embodiments of a handpiece for micro perforation of hard tissue surface.

FIG. 11 is a schematic illustration of one of the embodiments of a device for selective heating of hard tissue surface with a mouthpiece.

FIG. 12 is a schematic illustration of one of the embodiments of a device for treatment of hard tissue surface with melted solid-state material.

FIG. 13 is a schematic illustration of another embodiment of a device for selective heating of hard tissue surface with a hand piece.

FIG. 14 is a schematic illustration of yet another embodiment of a device for selective heating of hard tissue surface with a hand piece.

FIG. 15 is a schematic illustration of a process of laser micro-texturing of an enamel surface and selective heating of SMTL.

FIG. 16a is a schematic illustration of a process of laser micro-texturing of an enamel surface, impregnation by solid-state particles and selective heating to temperature T_{F}<Tₘₑₗₜ of hard tissue.

FIG. 16b is a schematic illustration of a process of laser micro-texturing of an enamel surface, impregnation by solid-state particles and selective heating to temperature T_{F}>Tₘₑₗₜ of hard tissue.

FIG. 16c is a schematic illustration of a process of laser micro-texturing of an enamel surface, impregnation by solid-state particles and selective heating to temperature T_{F}>Tₘₑₗₜ of hard tissue.

FIG. 17a is a schematic illustration of a process of laser micro-texturing of an enamel surface and impregnation by melted glass or crystals.

FIG. 17b is a schematic illustration of a process of laser micro-texturing of an enamel surface and impregnation by melted glass or crystals mixed with solid particles.

FIG. 18. Is a photograph of an area subjected to micro-texturing with an Er:YAG laser (a) before application of a composite material and (b) after application of composite material and de-bonding.

### DETAILED DESCRIPTION OF THE PREFERRED EMBODIMENTS

Hard Tissue Surface Preparation

The present invention is directed to a new method of tooth surface preparation. Specific to this method is the formation of a hard tissue surface microstructure, which is optimal for future application of dental materials, modification, sintering, changing of light back scattering propertiesy for better cosmetic appearance and other applications. The current method of acid etching creates a superficial porous layer with two types of the etched surface (Type I or II acid etching) or their mix (Type III). The method and apparatus of the present invention form the desired structure of the hard tissue surface structure by controlled optical patterns created by the energy of scanning microbeams or an array of microbeams.

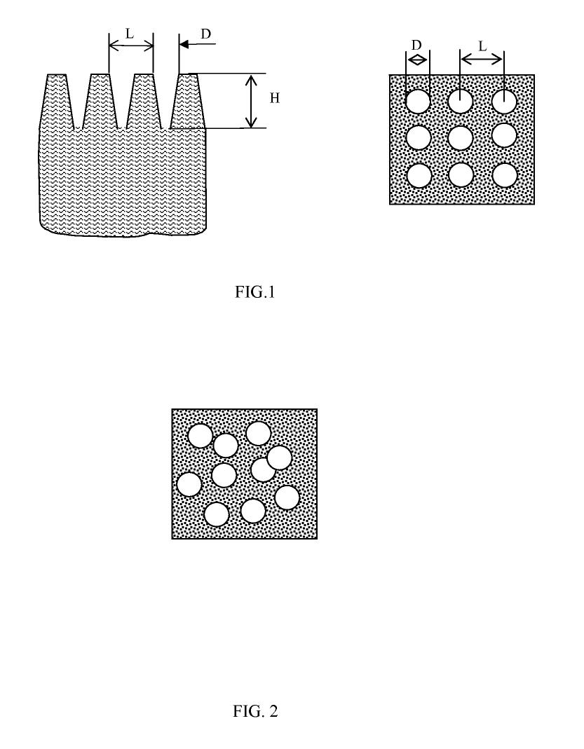

The method provides much more flexibility in adjusting the surface microstucture (microprofile) for future use. The formed microprofile includes micro holes or micro groves with a cross sectional size of D=0.1-250 μm and a depth of H=1-500 μm. The preferable cross sectional size is D=5-100 μm with depth H=3-50 μm. A typical ratio of the depth to the cross sectional size (aspect ratio AR) is 0.5 to 50 times. A regular (microhole) structure provides for more reproducible and uniform adhesion, bonding and sintering structure. The effective area of the adhesion surface after such treatment can be increased several times compared to the area of hard tissue prepared for bonding using known methods, which leads to the increase of the adhesive strength. These microholes or microgroves can create a periodical, one or two-dimensional structure (an array of microholes or microgroves) on the hard tissue with a period of L=0.1 -10 D. FIG. 1 shows an example of such a structure. The fill factor F is defined as the ratio of an area of microholes or microgroves on the surface to the full treatment area. For the periodical structure shown in FIG. 1, F=πD²/4L². The fill factor can vary in the range of F=0.05-1. The array of microholes can be non-periodical or random, as shown in FIG. 2. In this case, instead of the period L we use the average distance Lₐᵥ between the centers of the holes. Deep holes or groves with aspect ratio AR=1-50 can be created using this method, which is not possible to achieve with the known etching method. Such holes with AR>1 are redistributing the stress applied to the bonding surface and increase adhesion due to the “anchor” effect.

From the percolation theory it follows that if fill factor F>A, where A is a threshold of percolation, then all the holes form a continuous cluster. If F<A, then every hole is isolated from one another by walls and the residual part of the hard tissue creates a continuous cluster. The threshold of percolation A is approximately equal to 0.65-0.75. For this reason, F must be lower than A, where F=0.05-0.065, in order for some applications to keep the strength of the residual hard tissue structure after formation of the microholes or microgroves. Such application can be sintering or tooth surface microprofiling improving the light back scattering,

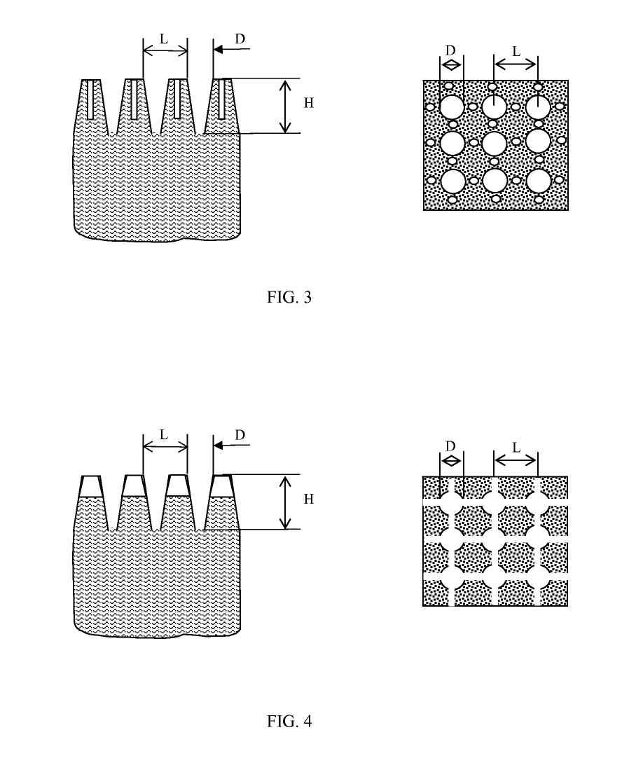

The geometry of the holes can be different, including, but not limited to, a cone, a circle, a cylinder, and a square cylinder. The axis of the hole can be perpendicular to the hard tissue surface or at an angle between 90° and 30°. As shown in FIG. 3, two and more periodical arrays of microholes can be created in the hard tissue. As shown in FIG. 4, one array can be an array of holes, while another can be in the form of groves (“bridges”) between the holes. If the depth of bridges is as small as 1-50 μm, then the fill factor of the entire micro structure can be F>A, without the significant decrease in strength of the residual hard tissue structure. The present invention is not limited to the described geometry and any other microstucture can be used with described geometrical parameters.

The structure described above can be formed on the enamel, cementum, dentine or bone surface. This structure can be formed on the bottom or the wall of a dental cavity. In addition, the microstructure can be formed on the surface of ceramic or veneers, composite, resin, implant and other artificial dental materials or structures.

Such a surface has significantly better adhesion and bonding properties to a dental material than that created after chemical etching due to a large contact surface and the deep holes. A dental material can penetrate into the holes and groves, creating additional protection against microleakage and secondary caries. Chemical etching can be applied after micro texturing for better removing of a smear layer and for increasing the contrast of the microstructure.

The proposed method and apparatus can be used for hard tissue surface modification for caries protection, hypersensitivity reduction, and esthetic improvement of the tooth. After the surface preparation described above, the holes can filled with different materials and affixed chemically, photochemically, therma-chemically or thermally. These procedures are described in detailed bellow.

FIG. 5. shows the holes and the bridge structure on hard tissue 501 coated by a dental material 502 and cured chemically, photochemically or thermochemically. The thickness of a layer of the cured dental material above the hard tissue can be adjusted by polishing. All existing adhesives can be used in combination with the hard tissue substrate preparation: glass-ionomers, one-step self etch adhesives, two-step self adhesives, two-step etch-and-rinse adhesives, three-step etch-and-rinse adhesives.

Microstructures on surface of the hard tissue can be formed by using the following energy sources, described below: mechanical, acoustical, and electrical or light. Chemical etching may be used in addition to the above treatments.

1) The mechanical method may use air or hydro abrasive energy. The flow of abrasive particles can be delivered through a small nozzle or an array of small nozzles. The flow of particles drills microholes in the hard tissue.

2) The acoustic method uses sources of acoustic energy, such as a transducer with an acoustic focusing system on the surface of the hard tissue. The focusing system focuses acoustic energy in one small focus or an array of acoustic foci. Acoustic energy is most effective in drilling microholes in cementum, dentine or bone.

3) The electrical method uses one microelectode or an array of microelectodes having the size of the tip between about 5-100 μm. The desired structure is formed in the hard tissue due to electro erosion.

4) Light energy is another method that can be used to form of the above-described microstructure on the hard tissue surface. Different types of lasers can be used for this propose. A laser having a ultra short pulse in the range of 1-1000 fs can be used for precise hard tissue perforation. The wavelength of this laser may be in the range of 100-20000 nm. A fluence on the tissue must be in the range of 0.0001-0.1 J/cm². A laser with a pulse width longer than 1 ps must have a wavelength that is highly absorbed by the hard tissue. The wavelength of the long pulse laser must be in the range of 100-350 nm or 1850-10,600 nm. Preferable wavelength must greater than the size of microstructures D and L. Such wavelength ranges are 100-250 nm, 2690-3000 nm and 9300-20000 nm. Fluence per microbeam with a pulsewidth longer than 1 ps must be in the range 0.01-200 J/cm² to provide ablation vaporization photoetching or modification of treated material. In the preferable embodiment the pulsewidth ranges is from about 0.1 μs to about 250 μs and the wavelength ranges from about 100 nm to about 350 nm, or from about 2690 nm to about 3000 nm, or from about 9300 nm to about 10600 nm, and the fluence in each microbeam is in the range from about 1 J/cm² to about 50 J/cm².

The most precise microstructure can be formed with wavelengths in the ultra violet (UV) range. Excimer lasers laser with non-linear converters can be used for this purpose. Er or Ho doped crystals can be used for the 2,690-3,000 nm wavelengths. An Er or a Ho doped fiber laser can be used for this range also. The CO₂ laser is the optimum choice for the 9,300 -10,600 nm range.

A handpiece for such treatment comprises several components, such as the laser or the output end of a delivery system from the laser mounted into the main box. The delivery system can be a fiber or an articulated arm. An optical system can be placed between the output laser or the delivery system and the treatment tissue to create an optical field for forming a microstructure on the hard tissue. The design of the optical system depends on the method of formation of the microstructure. Three different methods of treatment of the hard tissue for forming of the microstructure are the following

1) One microbeam is delivered to the tissue through the handpiece. This microbeam has a size close to D and fluence and pulse width necessary for ablating of the hard tissue. During treatment, the hard tissue surface handpiece and the microbeam can be moved across the hard tissue surface manually. The microstructure of the perforated holes is random as shown in FIG. 2. The optical system focuses a laser beam on the treatment surface or into the fiber tip having an output diameter smaller than D and with output end close to treatment surface. At the end of the tip can be mounting focusing system as microlense, sapphire ball or cone.

2) Spatial patterns such as an array of microbeams is delivered to the tissue through the handpiece to the tissue simultaneously. These microbeams have a size close to D. The periodical array has a period L and fluence and pulse width necessary for ablating the hard tissue. During treatment, the hard tissue surface handpiece is placed on the hard tissue and all microbeams are delivered to the tissue simultaneously. The microstructure of perforated holes or microgroves may be periodical, random or a combination of more than one microstructures shown in FIGS. 1, 2, 3, and 4. The optical system comprises of an optical element like spatial modulator with periodic modulation of the optical length (an array of microlength, phase mask, grating, diffractive optics, or holograms) or the coefficient of reflection (a minor or an array of micromirrors). MEMS-type reflectors can be used as a part of the optical system. The spatial modulator as array of microlenses or micromirrors can be illuminated by optical beam with the size smaller than size of the spatial modulator and can be placed near the tissue to provide array of microbeams on the tissue. In other embodiment spatial modulator is imaged (projected) on the surface of treatment tissue or material.

3) One microbeam is delivered to the tissue through the handpiece. This microbeam has a size close to D and fluence and pulse width necessary for ablating the hard tissue. During treatment, the hard tissue surface the microbeam may be moved across the hard tissue surface by a scanner. The microstructure of the perforated holes or groves can be periodical or random or as combination of more than one microstructures shown in FIGS. 1, 2, 3, 4. The optical system focuses the laser beam on the treatment surface. Microholes or microgrooves are drilled sequentially. The structure of the holes is determined by the scanner's algorithm. This algorithm can synchronized with the motion of the handpiece across of the hard tissue.

The handpiece can be equipped by contact and motion sensors, sensors for control of the hard tissue ablation process (acoustic, motion, thermal, spectral), an auto focusing system which keeps the position of the microbeam focused on the surface of the treated tissue, a system for cooling of the tissue, a laser and other components.

The pulse width must be shorter than 0.1-10 thermorelaxation times (TRT) of the treatment tissue. The TRT can be calculated using the following formula:

$$TRT \approx \frac{{D}^{2}}{16 \cdot α} , \qquad ( 1 )$$

where α is the thermal diffusivity. For the enamel

$${α}_{enaml} \approx 0.004 \frac{{cm}^{2}}{sec} .$$

The pulse width must be shorter than 1000 μs, preferably shorter than 100 μs and most preferably shorter than 10 μs. Several pulses can be delivered into the same microholes for deep drilling. The fluence for the microbeam with pulse duration in the 0.01-100 μs range must be in the range effective for ablation: 1-200 J/cm².

### EXAMPLE 1

### A Handpiece for Microperforation of Hard Tissue Surface

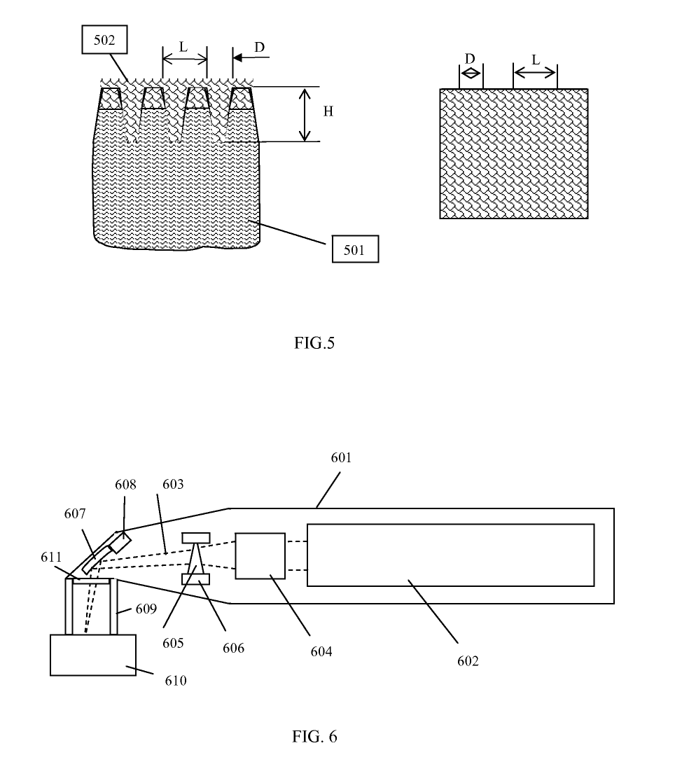

The handpiece is shown in FIG. 6 and comprises of a body housing 601, a laser 602, and an optical system 604. A microcomputer control scanner may comprise of an optical wedge 605 and a mirror 607 mounted on rotated on translated platforms 606 and 608, a spacer 609 and a protection window 611. A laser beam 603 is focused and moves across the treatment tissue or a material surface 610. The laser may be a diode laser, a solid-state laser pumped with diode laser or a fiber laser pumped with a diode laser, flash lamp pumped or gas laser. Single mode fiber laser is preferable embodiment due very high brightness and capability to create very small microbeam with minimum diameter. The laser may function in continuous wave (CW) or pulsed mode, synchronized with the scanning of the beam. Typical laser parameters are as follows: wavelength 2.7-2.94 μm; micro pulsewidth 0.1-100 μs. In addition, energy per pulse for perforating a hole with a diameter D=1-250 μm has to be in a range of 0.001-100 mJ. In another embodiments the scanner can be designed as mechanically translated, rotated or bent waveguide.

### EXAMPLE 2

### Adhesion Improvement after Laser Microtexturing

Extracted tooth samples were used in the following test. All samples were extracted from human subjects between the ages of 25 and 40 for periodontal or orthodontal reasons. Two areas, treatment and control, were selected on the opposite sides of each sample. Every treatment area was processed using laser micro-texturing or acid etching protocols described in detail below. A metallic rod was then attached perpendicularly to every treatment area using a dental composite material (Revolution, Kerr, USA). Another metallic rod was attached in a similar manner to every control area. Both areas were then subjected to the below-described bond strength test using a special device (ZIP, RMU-0.05-1, USSR) having a load speed of 50 mm/min.

Laser micro-texturing protocol. A total of 12 extracted tooth samples (8 enamel surfaces and 4 dentinal surfaces) were treated using the following protocol. Treatment area of each sample's was placed under an lens (f˜38mm) and was processed with the Er:YAG laser in free-running mode, having a wavelength of 2.94 μm and energy per pulse of about 1 mJ. A single laser treatment with parameters as described above without water spray formed a single crater having a diameter of about 100 μm. Thereafter, a matrix of 40×40 craters was formed on the treatment area using laser radiation. The distance between each crater was on the order of 50 μm. The linear size of the matrix was 2×2 mm. Following the laser treatment, the tooth surface was thoroughly rinsed under distilled water and alcohol for 30 seconds and dried with compressed air for 10 seconds.

Chemical modification protocol. A total of 20 extracted tooth samples (16 enamel surfaces and 4 dentinal surfaces) were treated using the following protocol. Each sample's treatment area, having the size of 2×2 mm, was filed with a diamond disk to the depth of about 100 μm. The surrounding surface was then covered with polish. The treatment area was then thoroughly rubbed with alcohol and dried under an air jet for about 20 seconds. Next, the area was etched with a self-etch primer (Nano-Bond Self-Etch Primer, Pentron, USA), dried for 20 seconds and exposed to compressed air for another 20 seconds. Thereafter, the area was covered with a Nano-Bond Adhesive (Pentron, USA), dried for 1 minute and then cured with an LED curing light (Allegro™ Rembrandt®, DenMat, USA) for 20 seconds according to the manufacturer's instructions.

The surface of each tooth sample's control area was treated according to the protocol of chemical modification described above, however, the overall treated area was greater than 20mm². Following the above described chemical modification and laser micro-texturing treatments, metallic rods, having a diameter of 2 mm, were attached to both the treatment and control areas by immersing the rods 3-4 mm into flowable composite (Revolution, Kerr, USA). The composite was cured for 30 seconds with the above-described LED curing light.

Next, a bond strength test was conducted to determine bond strength of studied surfaces in kgf. Given that the studied area was 4 MM², the corresponding bond strength magnitude was calculated in MPa and summarized in Table 1 below.

TABLE 1
Bond strength.

| | Modification | Bond strength, MPa |
|---|---|---|
| Enamel | Er: YAG laser | 21.88 ± 7.63 |
| | Nano-Bond Self-Etch Primer | 14.37 ± 6.23 |
| Dentine | Er: YAG laser | 21.56 ± 5.93 |
| | Nano-Bond Self-Etch Primer | 14.76 ± 9.23 |

It was observed, that the bond strength of areas treated with laser micro-texturing was 1.5 times higher than that of areas etched with phosphoric acid. Further study of debonded interfaces revealed that laser craters were filled with the composite as shown in FIG. 18. In addition, it was found in a separate test that microhardness of micro-textured enamel surfaces is non-uniform and increased in the area around microholes (FIGS. 18) to 1,070 kgf/mm² vs. microhardness of intact enamel of 380 kgf/mm². This surprising phenomena of laser micotexturing can be one of the mechanisms of increased bonding of the optical radiation strength.

Tooth Coating after Treatment with the Tooth Rejuvenation Compound

Immediately after treatment with the method and apparatus described above, the hard tissue can be covered with a coating, permeable to the important materials, which provide protective, prophylactic, therapeutic (for example, affecting the re-mineralization process) and/or esthetic effects. The materials include, but are not limited to the following direct and indirect restorative dental restorative materials: amalgam, conventional glass ionomers, resin modified glass ionomers, compomers, fluoride releasing resins, conventional composites, and ceramics (including high, medium or low fusing porcelains).

The protective coating would be impermeable to the majority of organic molecules, which would otherwise pigment the enamel after beaching. The porous layer of enamel after treatment with the compound is better suited for bonding of coating material with tooth structure. The adhesion mechanism of such material may include etch-and-rinse, self-etch or glass-ionomer adhesion. An example of such a coating material is BISCOVER™ compound (BISCO, Inc.), which is a light cured composite. The effective adhesion of this coating material to a tooth treated with citric acid at a pH=1.5 with temperature 50° C. for 5 min was demonstrated. The result was a tooth surface, which was resistant to mechanical abrasion and acid attack. The optical, mechanical and chemical properties of the coating material can be improved by adding particles with special properties. The addition of sapphire, diamond, fianite, granite, topaz, amethyst, quartz, crystal, zircon, agate, spinel, and heavy flint glass particles increases scattering properties of the coating due to great differences between refractive indexes of particles and polymerized matrix. Scattering efficiency is directly proportional to the square of the difference between refractive indexes of the particles and of the matrix. Typical refractive index of the polymer matrix ranges from 1.4-1.55. Any particles from solid bio-compatible material with a refractive index higher than 1.6 are suitable for this effect. In addition, these particles can improve the wear resistance of the tooth. The size of these particles can vary between 10 nm-50000 nm. The particles can be arranged in the form of a sphere, a plate, or a fiber. In one embodiment, the fiber can be woven into a mesh. The mesh can be incorporated into the coating compound, applied to tooth after treatment with tooth rejuvenation compound, and then polymerized. This fiber can be made of quartz, glass, or crystal.

In another embodiment, the hard tissue surface is impregnated by a liquid silicon glass after microtexturing. The above-described nano or micro particles can be added to the porous layer of the hard tissue or to the silicon glass. After drying of the liquid silicon glass in the porous layer, a modified layer of hard tissue with better mechanical, chemical and optical properties is formed. In addition, properties of this layer can be further improved by selective heating of this layer to the melting temperature of the silicon compound or of apatite, which is in the range of 1000-1200° C. Methods and apparatus for selective heating of this layer are described in detail below.

A special color center can be added to the coating material to provide a unique optical property to a tooth, e.g. ruby or alexandrite particles would produce a pink color. Gold, silver, or platinum particles could be added, as could organic dye molecules, which can be bleached at any time using UV light.

Nanoparticles (fullerenes or astrolenes), could be deposited immediately after cleaning. A solution of these particles penetrates the pores of the enamel and forms a thin film on its surface. Another coating of a material preventing the nanoparticles from diffusing into the environment surrounding the teeth is then deposited over the original thin film. The nanoparticles become locked in between the original thin film and the coating. Since it is known that the ability of nanoparticles to facilitate oxidation of the surrounding elements by generating singlet oxygen increases when the particles are exposed to light, the nanoparticles will oxidize the enamel of the tooth and bleach it more efficiently during the day when exposed to day light, and less efficiently at night. The effectiveness of such bleaching depends on the properties of the nanoparticles, their concentration as well as of the ability of the protective coating to diffuse oxygen, which should be sufficiently high.

Tooth Rejuvenation and Protection Due to Temperature Modification of the Tooth Surface

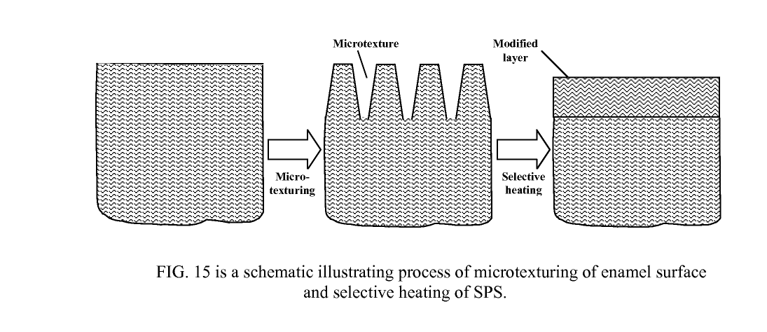

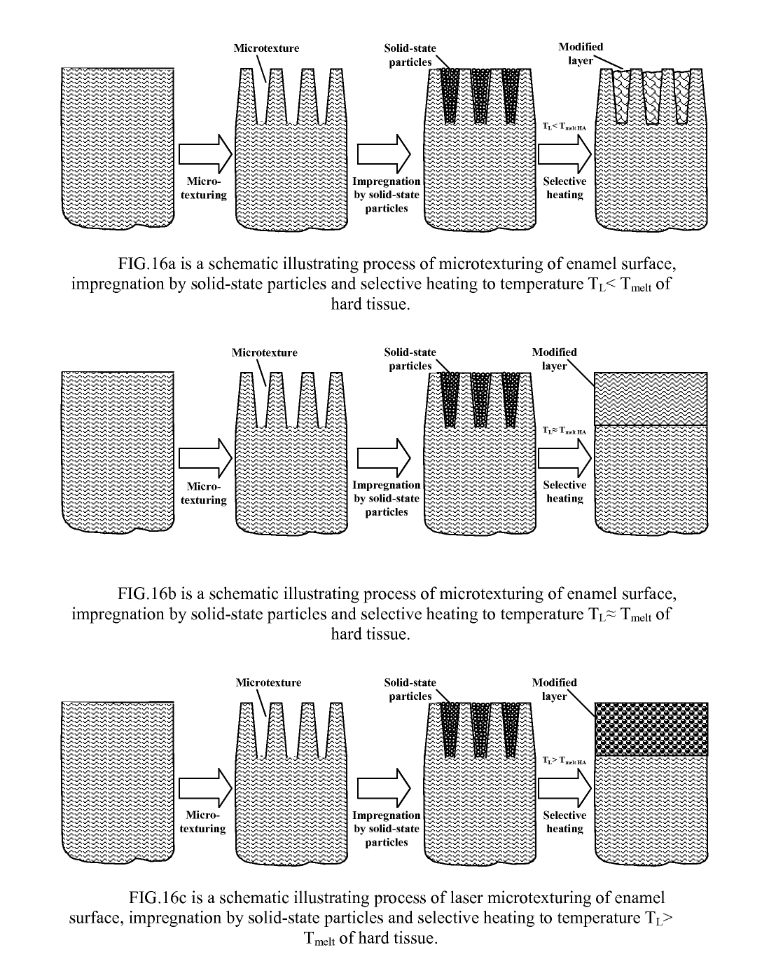

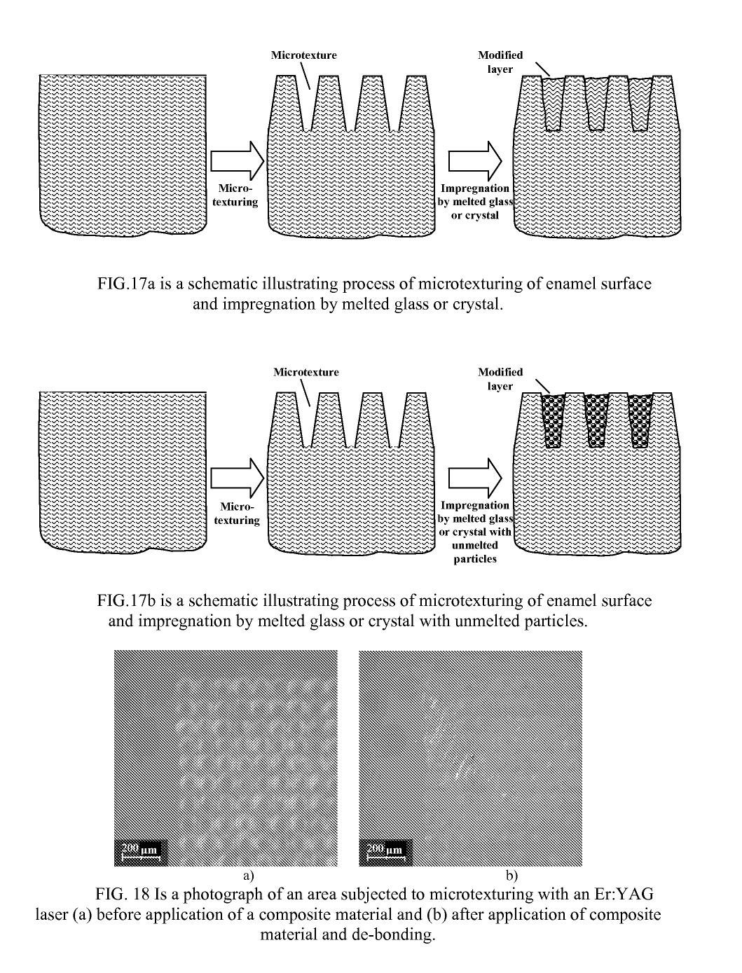

A method and apparatus for professional tooth surface rejuvenation and whitening using edible acids was proposed and described in the above sections. This method can be further improved by additional selective heating the tooth surface. A method and apparatus for such heat treatment, which is described below, can be applied to etched hard tissue surface, carious tissue, or dentine and cementum tissue. In addition, the heat treatment can be used for treatment of gingival recession. Gingival recession is exposure of the tooth's root surface, caused by a shift in the position of the gingiva. Recession may be localized to one tooth or a group of teeth and may be visible or hidden. Caused by such factors as improper tooth brushing, gingival inflammation and aging, gingival recession promotes tooth's susceptibility to caries, sensitivity and undesirable esthetic appearance. The main requirement to hard tissue surface for such treatment is that microtexture or superficial microtextured layer (SMTL) must exist on the surface. A preferable method of creation of the SMTL, proposed in this invention, is the formation of a regular microprofile on the hard tissue surface using a laser, the laser abrasive method or acoustic energy. Three groups of treatment are proposed in present invention: 1) a group of methods, based on the heating of the SMTL with subsequent recrystallization, amorphization or ablation of at least some portion of the SMTL layer (FIG. 15); 2) a group of methods based on the heating of the SMTL layer impregnated with solid-state nano and micro particles (FIG. 16); 3) a group of methods, based upon the impregnation of the SMTL by a preheated organic or mineral compound in the liquid phase (FIG. 17). After application of some or all of these methods, a superficial layer of hard tissue is formed. Such layer has enhanced optical properties, hardness and resistance to acid when compared with the original enamel or dentine. These methods can be used for tooth rejuvenation and protection, closure of carious lesions, treatment of hypersensitivity by sealing of dentine tubules, and treatment of periodontal disease. The three types of said treatment are described below.

Thermal Treatment of the Superficial Layer of Hard Tissue

The previous method of hard tissue treatment using edible acid alters the hard tissue structure, by the formation of a layer of SMTL with a depth varying from 0.5 to 500 μm, in a controlled manner. This change of hard tissue structure is accompanied by a deep cleaning of the surface of the hard tissue layer from staining, resulting into an improvement in tooth color. In addition, apatite crystals with micro-defects in the superficial layer of enamel are removed. Another effect of such treatment is removal of micro crystals with the lowest acid resistance, such as carbide apatite crystals. After such treatment, the surface layer of enamel is exposed to an intensive process of remineralization from saliva or other remineralizing rinses. However, the exposed layer of hard tissue can also be used for re-crystallization and the creation of a thin film of re-crystallized or amorphous apatite. This film has a higher acid resistance than natural hard tissue and additional light scattering properties, resulting in an improved aesthetic appearance of the tooth. It has been shown that the concentration of calcium (Ca), phosphorous (P) and fluorine (F) in the surface level of enamel is considerably higher than in that of the subsurface layer. The surface concentration of fluorapatite may be ten times more (10×) that of subsurface concentration of fluorapatite. However, the concentration of fluorapatite by weight is considerably less than that of hydroxyapatite. Under acid attack, the solubility of Ca ions in hydroxyapatite is considerably higher than the solubility of F ions. Therefore, the concentration of fluorapatite in modified enamel is increased considerably after acid attack. In the present invention, we propose laser post-treatment of the modified hard tissue surface layer as well as of the subsurface layer. Such treatment includes the selective heating of the modified surface layer as well of the subsurface layer to melting temperature, which ranges from 900° C. to around 1200° C. for enamel, and from 700° C. to around 900° C. for dentine. This is considerably lower than the evaporation temperature of these tissues, which is greater than 2000° C. After controlled cooling of the melted, modified hard tissue layer, a film is formed on the hard tissue surface in a crystallized or amorphous form. The film consists of crystallized or amorphous apatite, with a concentration of fluorapatite greater than that of the original enamel. This film improves the tooth's resistance to carious attack because: 1) an increased concentration of fluorapatite provides for a higher acid resistance against acids generated from biofilm or from foods; 2) the film has a higher density than regular enamel and is characterized by lack of defects and pores, which allow for penetration of bacteria and acids into subsurface enamel layers; 3) the film can function as a sintering surface for better post treatment remineralization from saliva or remineralizing rinses than for natural enamel. The film also has higher light scattering properties because the index of refraction for the re-crystallized layer is higher than the index of refraction of the subsurface layer of enamel due to a different chemical composition. Following re-crystallization, the surface layer is a glazed, minor surface, with minimal scattering properties. However, the border between the re-crystallized layer and subsurface layer is irregular, with typical size of said irregularities equal to the size of the enamel prisms (5 μm). Such a border has high scattering properties. Light scattering from this border prevents the penetration of light into the subsurface tissue and reduces the portion of light scattered from subsurface layers of enamel and dentine in the general volume of light scattered from the tooth. Therefore, the cosmetic appearance of the tooth is determined more by the scattering of light from the border between the re-crystallized layer and the subsurface layer. The re-crystallized layer does not contain color centers, as these centers are removed during acid treatment. Therefore, the light reflected from the re-crystallized layer and from the subsurface layer is perceived as white. At the same time, scattering from the inner layers of enamel, which may be colored due to change in organic components due to aging, accumulation of color centers, penetrating tooth externally or internally (e.g. tetracycline), is suppressed. Such treatment can enhance the hardness of tooth the surface using proper post-cooling, which is described in detail below.

The proposed method includes two steps: 1) the formation of a layer of SMTL on surface of hard tissue with a predetermined depth of 0.5-100 μm; 2) selective heating of the layer to a temperature ranging from 700 -2000° C. and controlled post-cooling of the layer to form crystallized or amorphous film of apatite on the tooth surface. Pulsed heating of the layer can be with preheating pulse, which elevates temperature of the layer and under layer of tissue to meting point and is followed by heating pulse, which selectively melts the porous layer (melting pulse). The preheating pulse width τₚᵣₑₕₑₐₜ can be greater than or equal to the thermal relaxation time (TRT) of the SMTL. Melting pulsewidth τₘₑₗₜ would be in the range of 0.1 TRT-10 TRT, preferably in the range between the TRT of the non-porous superficial layer and the TRT of the porous superficial layer. The TRT can be calculated using the formula:

$$TRT \approx \frac{{d}^{2}}{4 \cdot α} , \qquad ( 2 )$$

where d is the thickness of the layer d≈0.5-100 μm, and α is the thermal diffusivity. For non-porous enamel

$${α}_{enaml} \approx 0.004 \frac{{cm}^{2}}{sec} .$$

A porous layer with a porosity p has thermal diffusivity αₚₒᵣₒᵤₛ≈αₑₙₐₘₑₗ·(1−p)^{1/3}. The porosity of the enamel after etching and drying can be in the range 0.1-0.7. Based on the formula (2), the TRT of the porous layer can be in the range as shown in Table 2.

TABLE 2
Thermal relaxation time of the enamel layer in μs.

| Enamel layer thicknes [symbol] | Porosity | | | | |
|---|---|---|---|---|---|
| microns | 0 | 0.1 | 0.3 | 0.5 | 0.7 |
| 0.5 | 0.16 | 0.16 | 0.17 | 0.20 | 0.23 |
| 25 | 390.63 | 404.59 | 429.94 | 492.16 | 583.52 |
| 50 | 1531.00 | 1586.00 | 1686.00 | 1929.00 | 2288.00 |
| 75 | 3422.00 | 3545.00 | 3767.00 | 4312.00 | 5113.00 |
| 100 | 6064.00 | 6281.00 | 6674.00 | 7640.00 | 9058.00 |

[symbol]

indicates data missing or illegible when filed

The melting pulse width can be in a range from 16 ns to 90 ms. The preheating pulsewidth can be in the range from 160 ns to 90 ms. Cooling of the melted enamel or dentine layer is important for the formation of a new layer of hard tissue to provide better optical, mechanical and chemical properties. Rapid post-cooling leads to the formation of a mostly amorphous glass-like structure. Slow post-cooling leads to the formation of mostly a fine or coarse-crystalline structure. The crystalline structure may be more preferable for thick modified layer. An amorphous structure may be more preferable for a thin modified layer. For some applications, the modified layer can be formed with a deep crystalline structure and a thin superficial amorphous layer. Cooling can be passive or active. The tooth can be cooled by allowing heat to dissipate into the tooth structure (passive cooling) or the tooth can be cooled from the heated surface with a cooling gas or liquid (active cooling). For example, a water layer with a thickness ranging from 10 μm to 5 mm can be applied to the surface of the treated tooth with or after the melting pulse. In this case, heat is removed by thermoconduction to the water layer, leading to its heating and vaporization. Post-cooling may be beneficial to decrease the residual amount of heat remaining on the tooth after treatment. To extend the post-cooling time, a long post-heating pulse can be applied to the treated layer of hard tissue. The post-heating pulse duration can be from TRT of melted layer to 1 sec. Controlled post-cooling can prevent the formation of droplets on the surface during solidification. The amount of heating energy required for this treatment can be calculated using the formula:

F=d·ρ·(1−p)·(Q+c·ΔT), (3)

where ρ is the enamel density, c is the enamel-specific heat capacity, Q is the enamel-specific heat of melting, ΔT=Tₘₑₗₜ−37, Tₘₑₗₜ is the temperature to melt the hard tissue. The minimum fluence of heating energy for melting as a function of thickness of the porous layer and porosity is shown in Table 3.

TABLE 3
Fluence of heating energy for melting the porous layer of enamel in J/cm²

| Enamel layer thicknes [symbol] | Porosity | | | | |
|---|---|---|---|---|---|
| microns | 0 | 0.1 | 0.3 | 0.5 | 0.7 |
| 0.5 | 0.19 | 0.17 | 0.13 | 0.09 | 0.06 |
| 25 | 9.46 | 8.51 | 6.62 | 4.73 | 2.84 |
| 50 | 18.73 | 16.85 | 13.11 | 9.36 | 5.62 |
| 75 | 28.00 | 25.20 | 19.60 | 14.00 | 8.40 |
| 100 | 37.27 | 33.54 | 26.09 | 18.63 | 11.18 |

[symbol]

indicates data missing or illegible when filed

It follows from the above table, that the range of minimum heating fluence for the described method is Fₘₑₗₜ=0.06-37 J/cm². The fluence for this treatment is G times higher than Fₘₑₗₜ, where G is the inverse efficiency of absorption of the heating energy in the treated layer. For dentine treatment, the fluence is 2-4 times lower than that for enamel. Table 3 shows that porous tissue has a melting fluence 1.1-3.1 times lower than that of intact tissue. This property can be used for selective treatment of tissue processed with acid in such a manner as to not affect the untreated tissue. To do this, the fluence must be selected from the range of G·Fₘₑₗₜ<F<G·(1.1-3.1)·Fₘₑₗₜ.

The heating of the SMTL in the present invention can be achieved using several energy sources, including, but not limited to, electromagnetic energy sources, such as a laser, microwave generated sources, electrical current sources, such as direct current, low or radio frequency current sources, or acoustic sources.

One embodiment of present invention is shown in FIG. 10. The device comprises of a power supply 10-2, a control unit 10-3, a cooling unit 10-4 and an energy source unit 10-5. Unit 10-5, in turn, may comprise of one or more energy modules 10-6, each generating its own energy type (e.g. laser radiation, microwave, acoustic wave, high-frequency current, etc.). The main unit 10-1 is connected to a handpiece 10-7 by way of a flexible tube 10-8, which, in turn, may contain flexible tubes for the transmission of cooling liquid from 10-4 to the tooth surface 10-9 via a jet 10-10. In addition, the flexible tube 10-8 may contain optical fibers or hollow waveguide for transmission of laser energy and/or hollow waveguide for transmission of microwaves to 10-9 via the tip 10-11, and/or electric wires for supply of electrodes, and/or the acoustic transducer situated in 10-11. The tip 10-11 transmits to the tooth 10-9 one or several energy types. For transmission of laser energy, the tip 10-11 may be an optical fiber 10-12, fixated in holder 10-13. For transmission of microwaves, the tip 10-11 may be a hollow tube 10-14, fixed in holder 10-15. For creation of an acoustic wave on the surface 10-9, the tip 10-11 may be an acoustic transducer 10-16, fixed in a rod 10-17, which, in turn, is fixed in a holder 10-18. Energy is delivered to the acoustic transducer is done via wires 10-19. Electrodes 10-20 may be used for the creation of a high-frequency discharge on the surface 10-9. The distance between exposed electrode tips 10-20 may be between 0.1 mm and 1 mm. The electrodes 10-20 are situated in a rod 10-21. The rod is fixed in a holder 10-22. Energy is delivered to the electrodes 10-20 via wires 10-23. Holders 10-13, 10-15, 10-18 and 10-22 attach these structures 10-11 to a tip 10-7.

In addition, a sensor 10-10 for feedback-controlled treatment can be incorporated into the tip. The sensor can be used for differentiation of the porous layer from intact hard tissue or soft tissue, measurement of the temperature of the layer's, measure melting point, and measurement of contact with the tissue. This sensor can be mechanical, electrical, optical or acoustic. For example, it can be an IR sensor for measuring the temperature of the surface, as shown. The signal from the sensor is sent to control electronics 10-3 and is used to control the level and temporal profile of the heating energy. The shape of the tip 10-11 can be round, with a diameter of 0.05-3 mm, or rectangular. Two different types of energy can be combined for heating. For example, pre-heating and post-heating pulses can be microwave, electrical or acoustical pulses, while the melting a laser beam with a diameter once it reaches the surface, of 0.01-0.5 mm can be controlled by a micro scanner to produce uniform or predetermined non-uniform patterns on the tooth surface. This device can be used for treatment of all teeth. The treatment area can be controlled by the operator and moved from site to site by the hand of the operator. This device can be used for the selective treatment of fissures, dentine periodontal area, sharp edges of a tooth, and carious lesions. The device can also be used for preparation of tooth surface prior to application of filling or crown material and veneers. In this case, special surface profile can be created on the tooth's surface for better bonding. The modified layer of the tooth's surface can provide additional protection against recurrent caries, for periodontal decease prevention and healing, hypersensitivity treatment.

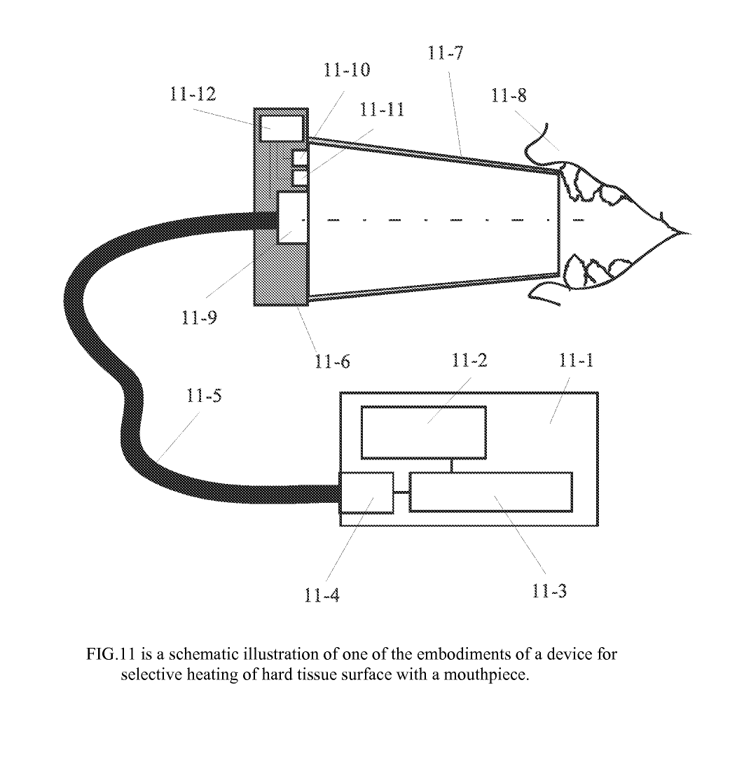

In another embodiment, the anterior teeth can be heated using an automatically scanning laser beam, as shown in FIG. 11. This device may contain a main unit 11-1 with a power supply 11-2, a laser with optics 11-3 and an optical coupler 11-4 into the fiber 11-5. Laser energy through the fiber 11-5 is delivered into a mouthpiece 11-6. The mouthpiece comprises of a body 11-7, held in the mouth 11-8 of the patient, an optical two or three-dimensional scanner with focusing optics 11-9, an optical video camera 11-10 and an optional thermo camera 11-11. Signals from the cameras are transferred to control electronics 11-12, which controls the scanning mode of operation of the scanner 11-9. The image from the cameras 11-10 and 11-11 can be presented on a monitor. The operator can use the image on the monitor for determining treatment areas, for programming the scanner, and for real time observation of treatment.

Laser sources for practice of this invention can be selected from those lasers with energy and pulse width described above, and wavelengths, which are primarily absorbed in treated layer of hard tissue. In preferable embodiment, laser light penetration into the hard tissue must be close to or lower than the thickness of the treated layer, which for this invention is in the range of 0.5-100 μm. The depth of penetration in the tissue is expressed by the formula, h=1(μ_{abs}(λ)+μscatt(λ)), where μ_{abs}(λ) and μ_{scatt}(λ) are the coefficient of absorption and the coefficient of scattering of the tissue as a function of the wavelength λ, respectively. For h=(0.5-100) μm, (μ_{abs}(λ)+μ_{scatt}(λ) is approximately (20000-100) cm⁻¹. Such strong absorption of enamel is found in the wavelength range λ=1.85-11 μm, preferably λ=2.7-3 μm and λ=8.7-11 μm, and most preferably λ=9.1-9.7 μm. Strong absorption and scattering of enamel is for the wavelength λ=0.15-0.4 μm, preferably λ<0.2 μm. In porous enamel or dentine in this range of wavelengths, the coefficient of absorption can be several times higher than in non-porous tissue due to the optical resonance (Mi resonance) on small particles in a porous structure. For the IR range of wavelengths Er, CO₂, CO, quantum cascade diode lasers, a fiber laser with diode laser pumping and optical parametric oscillators (OPO) can be used. For the UV range, excimer laser, solid-state lasers and a diode laser with a non-linear converter can be used. For example, a diode pumped Nd laser can be used with a 3, 4 or 5 wave non-linear converter. The laser can be built either into the main unit or into the handpiece. In another embodiment, one part of the laser system can be built into the main unit and another into the handpiece. For example, the Nd laser can be built into the main unit and laser energy can be delivered to the handpiece through an optical fiber. The non-linear converter can be built into the handpiece for direct delivery of UV light to the treatment zone. The lasers are described in greater detail below.

Heating of the SMTL Impregnated by Solid-State Nano and Micro Particles

In another embodiment of present invention, the superficial microtextured layer (SMTL) on hard tissue is filled with nano or micro particles and selectively heated to a temperature at which at least one component of the impregnated porous layers is melted to create a ceramic layer on the hard tissue surface after cooling. This method includes three steps as described below and shown in (FIG. 16):

1) Using the method and apparatus described in present patent superficial microtextured layer (SMTL)) of hard tissue with a thickness of 0.5-500 μm is formed on the tooth surface. SMTL is a layer with regular porous microstructure. A carious lesion or dentine surface with open dentinal tubules can also be considered as a microtextured layer and treated in this manner.

2) Solid particles, with size smaller than the size of the superficial microtextured layer, are impregnated into the porous structure using one of several conventional methods, such as painting of the suspension of the particle on the surface, application under pressure, etc.

3) The SMTL with the particles is selectively heated to a temperature sufficient to create strong bonding between the atoms of the particles and the atoms of the porous structure of hard tissue using the heating methods and apparatuses described above.

The size of the pores in the hard tissues prior to microtexturing, and after microtexturing is within the range of 10 nm to 5000 nm. Dimensions of micro texturing can be in the range 0.1-250 μm. The particle size must smaller than dimension of microtexture within this range, preferably within 5 nm to 100 μm. After heating of the porous layer impregnated with solid particles, at least one of the components is melted and, after solidification of the weak superficial layer, is replaced by a dense ceramic-like layer coating. The optical mechanical and chemical properties of this new layer can be optimized as necessary by selection of the type of the particles to be used. For example, by using particles with hardness greater than that of enamel it is possible to improve the wear properties of a tooth. Similarly, by using particles with a refractive index very different from apatite, scattering reflection and therefore, strong permanent whitening effect can be achieved. It is possible to create a ceramic with an acid resistance much greater than that of enamel or dentine. The ceramic-like layer is strongly bonded to the tooth because it is formed from to the tooth's porous layer, which is part of tooth's structure. This method can provide an improvement to the appearance of a tooth, better than is currently provided using veneers, with the significantly added benefit of not removing hard tissue or needing local anesthesia.

During the heating of layer of the SMTL impregnated with particles, at least one of the components of this layer must be melted and liquefied to a viscosity low enough to fill the pores. The dynamic viscosity of this heated component must be below η_{F}=10 Pa·s, preferably in the range of 1 to 0.0001 Pa·s. The temperature, when the solid state after melting exceeds this viscosity, is defined as the fluidity temperature T_{F}. For crystals, T_{F} is almost equal to the melting temperature T_{F}˜Tₘₑₗₜ. For glass, T_{F} is higher than temperature required to melt glass Tₘₑₗₜ, T_{F}=Tₘₑₗₜ+(100÷500). The T_{F} for glass-like composition can be calculated by the following formula:

T_{F}≈E/[R·ln(η_{F}/η₀)], (4a)

, where E is activation energy, η₀ is pre-exponential factor, and R=8.3 J/mol·K. The T_{F} can be also calculated by the following formula:

T_{F}≈{[(T₁−T₂)/T₁·T₂)/T₁·T₂]·[ln(η_{F}/η₁)·ln(η₂/η₁)]+1/T₁}⁻¹, (4b)

, where T_{1,2} is a temperature, when viscosity is η_{1,2} respectively. T_{1,2} can be transformation temperature (η=10^{11.3} Pa·s), softening temperature (η=10^{6.6} Pa·s) or melting temperature (η=10 Pa·s).

The present invention proposes the use of three types of particles.

1) Particles with a fluidity temperature T_{F}, lower than the temperature of melting of hard tissue (FIG. 16a), which is in the range of 1000-1200° C. for enamel and in the range of 700-900° C. for dentine. Therefore, T_{F}<1000° C. for enamel and T_{F}<700° C. for dentine. In this case, only the particles will melt and the SMTL will not change during heating. The melted particles will fill the pores of the hard tissue and fuse with it, bonding to the tissue. One advantage of this method is the low energy needed for heating, which results in a low cost of device. A lower temperature is also better for the tissues of the pulp and allows for very good bonding to the hard tissues. In the preferred embodiment, the coefficient of thermal linear expansion (CTLE) of the particles must be above that of apatite (CTLE=9·10⁻⁵) and below that of hard tissue. This would improve the strength of the bond during cooling and compress the composite/ceramic layer, avoiding micro cracks. The particles to practice this method can be organic, such as polymethylmethacrylate (PMMA), polycarbide, epoxy, etc. They could also be made of glasses, from the group of fluoride, phosphate, lanthanum or silica glasses. The fluoride glasses with a composition, such as ZrF₄—BaF₂—LaF₃—AlF₃—NaF, have a T_{F}=490 -800° C. Silica glasses with a compositions, such as Li₂O—SiO₂ or Na₂O—SiO₂, have a T_{F}=440-500° C. or T_{F}=360-410° C., correspondingly. Also crystals, such as Ca(NO₃)₂ (Tₘₑₗₜ=560° C.), Ca(OH)₂ (Tₘₑₗₜ=500° C.), BaO₂ (Tₘₑₗₜ=450° C.), CdCl₂ (Tₘₑₗₜ=570° C.) and others can be used to practice this invention.

2) Particles with a fluidity temperature T_{F} in the range of the melting temperature of enamel 1000° C.<T_{F}<1200° C. or dentine 700° C.<T_{F}<900° C. (FIG. 16b). In this case, both the particles and apatite are heated to the melting temperature, and are allowed to cool, creating an amorphous or polycrystal-like structure (composite/ceramic structure), depending on the heating and cooling regime used (described in detail above). The advantage of this method is the uniformity of the new composite/ceramic structure produced, and its high acid resistance. In the preferable embodiment, the CTLE of the new composite/ceramic layer must be lower than that of apatite (CTLE−9.10⁻⁵), thereby compressing the composite/ceramic layer and avoiding micro cracks during cooling. For this method, the particles used must be mineral, non-organic particles, such as glass or crystal, or a mixture of both. For example, the glass may have a composition such as Na₂O—Al₂O₃—SiO₂, and the crystal, a composition such as Ca(PO₃) (Tₘₑₗₜ=984° C.) or CdF₂ (Tₘₑₗₜ=1072° C.).

3) Particles with a fluidity temperature T_{F} in the range higher than the melting temperature of enamel (T_{F}>1200° C.) or dentine (T_{F}>700° C.) (FIG. 16c). In this case, after heating, the porous layer is impregnated with particles heated to a temperature higher than their temperature of fluidity T_{F}. The new structure is similar to the one described above. However, if the temperature is higher than the melting temperature of hard tissue but below the melting temperature of the particles, the composite/ceramic layer would be composed of solid particles bonded to the amorphous or crystallized apatite. One advantage of this method is the very high hardness of the new layer. In the preferable embodiment, the CTLE of the new composite/ceramic layer must be lower than that of apatite, compressing it, thereby avoiding micro cracks formation would be avoided during cooling. Lithium glass Li₂O—B₂O₃, for example 20Li₂O—80B₂O₃, with very low CTLE can be used to practice this invention. In this case, the particles to practice this method must be mineral, non-organic particles, such as glass or crystal and/or their mixture. Examples of appropriate glasses are quartz glass and sital glass. Glass with compositions, such as (Na₂O, CaO, SiO₂), (Na₂O, PbO, SiO₂), (Al₂O₃, Na₂O, SiO₂) (Na₂O, B₂O₃, SiO₂) can also be used. Examples of crystal are crystal quartz (Tₘₑₗₜ=1700° C.), diamond (Tₘₑₗₜ=3900° C.), sapphire Al₂O₃ (Tₘₑₗₜ=2046° C.), AlPO₄ (Tₘₑₗₜ=2000° C.), or CaTiO₃ (Tₘₑₗₜ=1960° C.), hydroxyapatite Ca₁₀(PO₄)₆(OH)₂ (Tₘₑₗₜ=1614° C.), fluorapatite Ca₁₀(PO₄)₆F₂ (Tₘₑₗₜ=1612-1680° C.). These crystals can also be chosen from the group of gem crystals, including, but not limited to, topaz, amethyst, zircon, agate, granite, spinel, fianite, tanzanite, and tourmaline. The particles can be made from high temperature ceramic and polycrystalline. The properties of some preferable particles used to practice the present invention are shown in Table 4. The T_{F} was calculated using formula (4).

TABLE 4
Material of the particles and their properties.

| | | Temperature of |
|---|---|---|
| | | melting Tₘₑₗₜ |
| Material | | or fluidity |
| Name | Composition, % | T_{F}, C. ° deg [symbol] |
| Diamond | C | 3700-4000 |
| Sapphire | Al₂O₃ | 2040 |
| Hydroxyapatite [symbol] | Ca₁₀(PO₄)₆(OH)₂ | 1614 |
| Quartz crystal | SiO₂ | 1610-1720 |
| Sheelite | CaWO₄ | 1580 |
| Fluorite | CaF₂ | 1418 |
| Glass | 50BaO—50SiO₂ | 1670 |
| | 50CaO—50SiO₂ | 1600 |
| | 28.4MnO—29Al₂O₃—38SiO₂ | 1600 |
| | 25MgO—25CaO—50SiO₂ | 1500 |
| | 50SrO—SiO₂ | 1460 |
| | 50Li₂O—50SiO₂ | 1350 |
| | 50PbO—50SiO₂ | 1100 |
| | 30Na₂O—10CuO—60SiO₂ | 1100 |
| | 19.7Na₂O—10.6Al₂O₃—69.7SiO₂ | 1050 |
| | 30Li₂O—18B₂O₅—52SiO₂ | 940 |
| | 50Na₂O—50SiO₂ | 900 |
| | 9Na₂O—38.7PbO—52.3SiO₂ | 850 |
| | 25.3Na₂O—53.6GeO₂—21.1SiO₂ | 650 |
| | 50K₂O—25TiO₂—25SiO₂ | 600 |

[symbol]

indicates data missing or illegible when filed

Dental ceramic composition (porcelain) can be used as particles to fill porous layer of the tooth. Low fusing dental porcelain frit, such as 68.6SiO₂-8.4Al₂O₃-1.84CaO-7.82K₂O-4.66Na₂O—0.1TiO₂-7.87B₂O₃-0.07Fe₂O₃-0.01Li₂O, with fusion temperature 850/1050° C. can be used as the first or the second type of particles. Medium fusing dental porcelain frit, such as 64.7SiO₂—13.9Al₂O₃—1.78CaO—7.53K₂O—4.75Na₂O—0.05TiO₂—7.28B₂O₃—0.07Fe₂O₃-0.01Li₂O, with fusion temperature 1050/1200° C. can be used as the second or the third type of particles. High fusing dental porcelain frit, such as 62.7SiO₂—17.1Al₂O₃—1.72CaO—6.94K₂O—4.245Na₂O—0.02TiO₂—6.92B₂O₃—0.07Fe₂O₃—0.01Li₂O, with fusion temperature 1200/1450° C. can be used as the first or the third type of particles.

Using gem crystals, the coating can create an entirely new appearance of the tooth, by controlling its color. For example, by using ruby crystal particles, the tooth would acquire a pink tone, with tanzanite or natural sapphire, a blue tone, while tourmaline would create a green tone. Diamond particles provide maximum scattering effect due to very high refractive index (n=2.5). Color of the coating can be adjusted by addition of small amounts of chromophore, such as Co or NaI, colloidal metal, such as Au, Ag, Pb, As, Sb, or Bi, semiconductor quantum dots, such as CdS, CdSe, CdTe, or ZnS. Photosensitive glasses, containing Au, Ag, Cu or other ions, can be used to provide color or darkness of the tooth, which is changes, depending upon light expose or temperature. In addition to dielectric particles, metal particles, including, but not limited to, Au, Pt, Ag, Cu or Ce could also be used. These particles would provide a unique cosmetic appearance and good wear and acid resistance to the tooth. These particles can be used for increasing selective absorption of the porous layer by laser heating or by changing of electrical properties of the layer by selective electrical heating. For example, adding Ce ions can increase absorption of the layer in the UV wavelength range. Selective heating of the porous layer, impregnated with nano or micro particles, can be achieved with light, microwave, electrical current and acoustic energy using the methods and apparatuses described in previous sections. Energy can be selectively deposited not only in the porous hard tissue layer, but also within the particles, which can be selectively heated to their melting point. This can for example be achieved using a laser. The wavelength of the laser must be selected from within the range where the ratio of the coefficient of absorption of the particles to the coefficient of absorption of the hard tissue is more than 2, preferably more than 10. The pulse width can be shorter than the TRT of the particles or their clusters, while the fluence is determined by equation (3). Due to optical or plasma resonances, it is important that the coefficient of absorption of the nano and micro particles can be significantly higher than that of the bulk material. The laser fluence can then be decreased, providing better safety of treatment and a lower cost of device. Lasers in the visible and near infrared range can be used for selective heating of the particles.

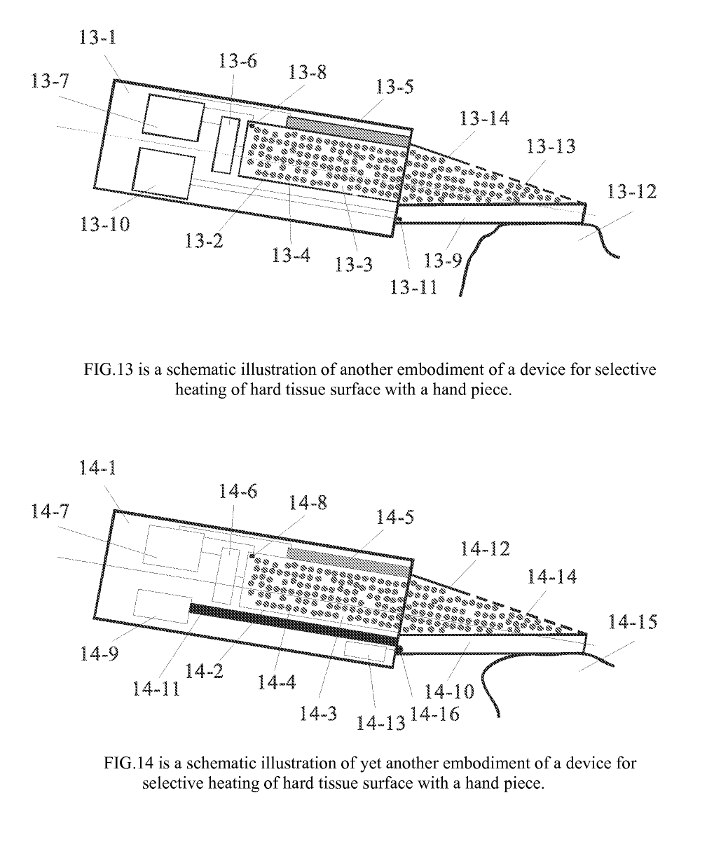

FIG. 13 shows yet another embodiment of the device, comprising of a probe 13-1, reservoir with the mixture 13-2 (e.g. in the form of gel) of a water-based acid solution 13-3 (e.g. using citric acid) and solid-state particles 13-4 (e.g. sapphire, diamond, etc.), a heater 13-5 for the mixture 13-2, a device to expel the mixture 13-6, a power supply and control unit 13-7, and a temperature sensor 13-8 of the mixture 13-2. The device also contains a heater 13-9 connected to the power supply and control unit 13-10. The temperature of the heater 13-5 is controlled by a sensor 13-11. A heater 13-5 is used for heating the mixture 13-2. Another heater 13-9 is used for melting of the modified hard tissue layer 13-2 by the tooth rejuvenation compound 13-3, which contains solid-state particles 13-4. The mixture 13-2 is delivered to the enamel upon contact of one side 13-13 of the tip 13-14 with the enamel. Heating of the modified enamel layer to the melting temperature occurs on contact of the heater 13-9 with the layer.

FIG. 14 shows one embodiment of the device, comprising of a probe 14-1, a reservoir with the mixture 14-2 (e.g. in the form of a gel) of the water-based acid solution 14-3 (e.g. using citric acid) and solid-state particles 14-4 (e.g. sapphire, diamond, etc.), a heater 14-5 for the mixture 14-2, a device for expelling the mixture 14-6, a power supply and control unit 14-7, and a temperature sensor 14-8 of the mixture 14-2. The device also contains a laser energy source 14-9, connected to a scanner 14-10 by an optical pathway 14-11 (e.g. optical fiber). The scanner is situated in the tip 14-12 and connected to the power supply and control unit 14-13. The mixture 14-2 is delivered to the enamel upon contact of one side 13-14 of the tip 13-12 with the enamel 14-15. The laser radiation transforms the enamel layer, modified by the acid, upon contact of the scanner 14-10 with said layer. The device also contains a contact sensor 14-16 connected to the power supply and control unit 14-13.

Impregnation of the SMTL by the Preheated Compound in the Liquid Phase

In another embodiment of the invention, the superficial microtextured layer (SMTL) on the hard tissue is filled by a compound preheated to liquid phase. At body temperature, the compound is in the solid-state phase. The melted compound impregnates SMTL of hard tissue and creates a ceramic layer on the hard tissue after cooling. This method takes up to three steps (the second step is optional) (FIG. 17):

1) Using the tooth rejuvenation compound based on an edible acid or other acid in the controlled manner described above, a porous layer of hard tissue with thickness of 0.5-100 μm is formed on the tooth surface. The surface could also be carious lesion or dentine with open dentine tubules.

2) (Optional) Solid-state nano or micro particles, with a size smaller than the size of the pores (10-5000 nm), are impregnated into the porous structure using one of several conventional methods, such as painting of suspension of the particles, application under pressure, etc.

3) The solid-state particles or a fibrous thin film of material are heated to the fluidity point T_{F} in close proximity to the tooth surface and are impregnated into the porous structure using external pressure or capillary power. The cooling phase begins after impregnation of the porous structure by the hot liquified material. During the cooling phase, if the T_{F}>Tₘₑₗₜ of enamel (800-1200° C.) porous enamel can be partly or completely melted and formed into a ceramic layer (FIG. 17a). If the T_{F}>Tₘₑₗₜ, after cooling, a heterogeneous structure of the SMTL filled with the solidified material is formed. If the second step is taken, the properties of the new layer can be optimized by changing the type of particles in this step. For example, if these particles have a melting temperature higher than T_{F}, then after cooling they are not changed and can provide the new layer with high hardness and good light scattering properties. Sapphire, ruby and other group of gem crystals, ceramic, or quartz crystal may be used. The particles may also be mixed with a low melting glass or crystal prior to delivery to the tooth surface (FIG. 17b).

The liquified material can be delivered to the SMTL under pressure for better impregnation. Alternatively, the liquified material can impregnate into the SMTL under the action of capillary pressure. Penetration coefficient of the liquified material must be maximized by selection of material with high surface tension, low contact angle (good wetting) and heating to the temperature higher than fluidity temperature For superior mechanical properties of the new layer, during compression of this layer, the compressive forces must be applied in a direction perpendicular to the tooth surface during the cooling phase. This compression can occur if the solid phase of the material has a lower density than the liquid phase. For example, a glass from the group of sital, CrO₂, CdS can be used. The cooling phase can be passive, by conduction into the deeper tissues or enhanced by surface cooling using a gas or liquid flow.

In another embodiment, a thin film of glass can be applied to the tooth surface. The thickness of such film can range between 5-100 μm. The film can be pre-cut to match contour of the tooth. Such film is soft and can be attached to the tooth surface by slight pressure. After that, the film can be heated to temperature T_{F} as are described above.

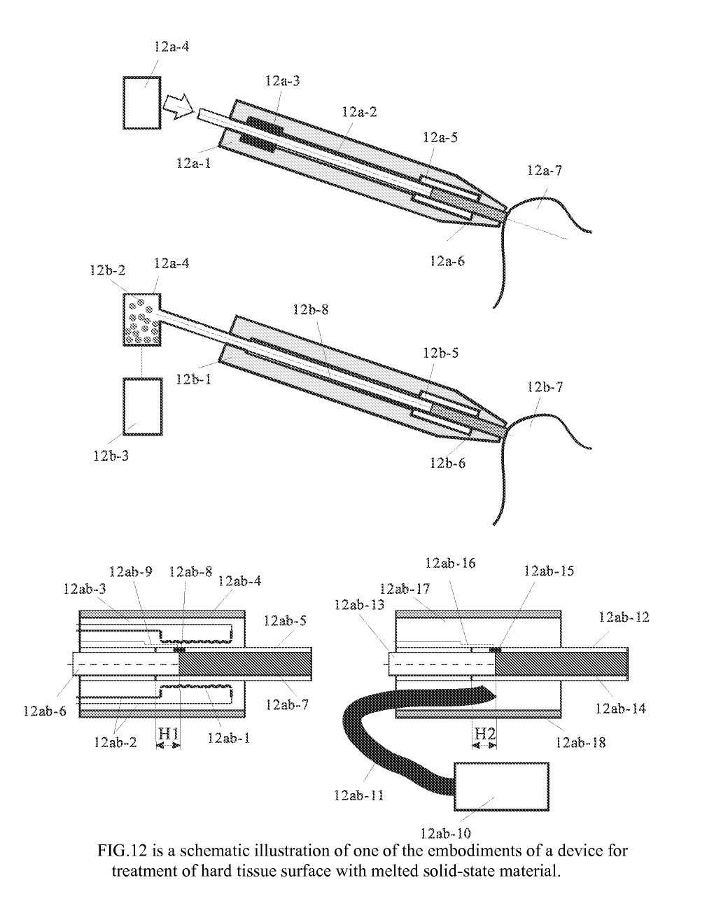

One embodiment is shown in FIG. 12. It comprises of a hand piece 12a-1, which contains a moving fiber 12a-2, made of sapphire, quarts, ceramic, fluoride glass, etc. The movement is accomplished by a mechanism 12a-3. The fiber is contained in a coil or container 12a-4. The device also contains a heater 12a-5, inside of which the fiber is melted. From the heater 12a-5, the melted material 12a-6 of fiber 12a-2 is delivered onto tooth enamel 12a-7 under pressure provided by the mechanism 12a-3. The heater 12a-5 can be one of the following: an electric heater, a non-coherent light source, a laser, a microwave source, an acoustic transformer, or a high-frequency electric current source, and a gas burner.

In yet another embodiment, shown in FIG. 12, the devices comprises of a hand piece 12b-1, which contains a tube 12b-8, along which solid-state particles 12b-2, such. sapphire, quartz, ceramic, fluoride glass, etc., move freely under pressure from the source 12b-5, which acts upon the particle container 12b-4. The device contains a heater 12b-5, inside of which melting of particles takes place. The melted material 12a-6 from the particles 12b-2 leaves the heater 12b-5 at a high speed and is delivered to the tooth enamel 12b-7. The heater 12a-5 can be one of the following: an electric heater, a laser, a microwave source, an acoustic transformer, or a high-frequency electric current source.

The heaters 12a-5 or 12b-5 can be electric heaters. An electric heater can be made from the wire fragment 12ab-1. An electric current is supplied to the wire fragment 12ab-1 via wires 12ab-2. The wire fragment 12ab-1 and partially wires 12ab-2 are placed in a thermo-insulated case 12ab-3 which is enclosed in another case 12ab-4 of the tip 12a-1 and 12b-1. The temperature of fragment 12ab-1, which is heated by current, is controlled by a change in its resistance. The heat generated by the fragment 12ab-1 via walls of tube 12ab-5 reaches the material of the fiber or particles 12ab-6. At a distance H1, from the entrance to the tube 12ab-5, the material of the wire and particles is melted, reaches tooth's surface 12a-7 (or 12b-7) via a tube 12ab-5 in a melted state 12ab-7. The temperature in the melting zone of the material 12ab-6 is controlled by a sensor 12ab-8, connected by wires 12ab-9 with the control unit of the device.

If the heater 12a-5 or 12b-5 is based on a laser, then the laser radiation source is 12ab-10. Laser radiation, conducted via an optical system 12ab-11, such as an optical fiber, reaches the tube 12ab-12 and is directed to the material of the wire or particles 12ab-13 via the walls of the tube. At a distance H2 from the entrance to the tube 12ab-12, the material of the wire and particles is melted and reaches the tooth surface 12a-7 (or 12b-7) in a melted state 12ab-14 via the tube 12ab-12. The temperature in the melting zone of the material 12ab-13 is controlled by a sensor 12ab-15, connected by wires 12ab-16 with the control unit of the device. The optical system and the tube are placed in a case 12ab-17, which, in turn, is situated in the case 12ab-18 of the tip 12a-1 (or 12b-1).

In the above embodiments, the distances between the heating zone and distal end of the contact tip is minimum in order not to cool down the melted fiber or particles, but sufficient to thermo-isolate the heater from the tooth. The method and apparatus described in this section is safer for tooth than direct heating because heating energy is applied to the filled material into the hand piece and not directly to the tissue. The rate of displacement of the melted compound is in the range of 0.1-1 mm³/s.

In practicing this method, after impregnating the SMTL by liquified material, a modified, melted layer is formed, which may not be as even as the original enamel layer. The resulting unevenness may be corrected by a rotary, polishing instrument, which is outside of the scope of this invention.

The present method and apparatus for modification of hard tissue surface can also be used for repair or improvement of ceramic or composite fillings, crowns, veneers and implants.

All of the devices shown in FIGS. 10, 11, 12, 13, and 14 are provided with tooth safety features. The major safety risk with heating of a tooth is thermal damage to the pulpal tissues. Pulp damage occurs when the temperature of the pulp exceeds 45° C. for a short period of time and 42° C. for a longer period of time. To prevent overheating of the tooth pulp several methods and features are proposed in present invention:

The total amount of heating energy and average power, deposited on a treated tooth, is limited, and can be calculated using the formula:

$${P}_{max} ≅ \frac{4 \cdot Δ T \cdot c \cdot ρ \cdot V \cdot α}{{δ}^{2}} , \qquad ( 5 )$$

where ΔT is temperature required to overheat the pulp (ΔT≈5° C., V is the tooth volume, and δ is the tooth thickness. Using the formula (4), the maximum average power of heat deposition on the tooth surface is approximately 0.3W.

A cooling agent, such as gas or air-cooling, is applied to the tooth surface to remove part of the heating energy. The cooling agent can be directed at the treatment zone or to the area surrounding the treatment zone. When using cooling, the maximum power Pₘₐₓ may be ten times greater than when not using cooling.

A temperature sensor could be used to monitor the temperature on the tooth surface and, based on this temperature, the heating energy and power can be controlled.

The method and apparatus for modification of dental hard tissue is not limited to dental hard tissue. The method and apparatus can also be used for treatment of other hard tissue in the human body and body of any mammal and animal. For example, the method of increasing chemical and wear resistance can be used in orthopedic surgery to improve such properties of a joint. In another embodiment, the method and apparatus can be used to improve wear resistance and aesthetic appearance of nail tissue. In practicing this method, a porous layer is first created on nail tissue using the above-described process of controlled etching by an acid based compound. The porous layer is then impregnated by solid-state nano and micro particles and heated to form a ceramic layer as previously described. The resulting ceramic layer has better mechanical and aesthetic properties than the original nail surface.

Improvement of Tooth Cosmetic Appearance after Micro Texturing.

The method and apparatus for modification of hard tissue can be used to improve aesthetic appearance of teeth and other organs. It is well known that optical surface scattering, together with superficial bulk scattering and absorption, contributes to the visual appearance of the teeth. Laser surface texturing can create regular or irregular scattering patterns and therefore created surface with controlled scattering properties, including intensity, angular and spectral distribution of scattered light. Moreover, regular pattern on the surface can serve as a diffraction grating and therefore provide spectrally selective reflection and scattering, thus modifying perceived color of the tooth. The surface texturing can be performed on external (labial or buccal), immediately visible surfaces of the teeth, or on surfaces being prepared for porcelain or composite veneer placements. In the latter case thinner veneers can be used because of more light scattering or otherwise modified adhesive surfaces. Therefore, less amount of the tooth material will be removed, resulting in less invasive technique and better adhesion between tooth and veneer.

Recording Pictorial and Digital Information on Hard Tissue

The method of hard tissue surface modification can also be used for recording non-uniform distribution on optical properties of tooth surface, including, but limited to spatially modulated coefficient of scattering, refractive index, coefficient of absorption or fluorescence property. One of many purposes of such modulation is to create a picture for esthetic proposes, including, but limited to a tooth tattoo, or to record and store information, including, but not limited to text, numbers, an informational picture or a hologram. The novelty of this method is with tooth enamel being just one example of hard tissue of the human body where information can be recorded and stored for a long period of time. As one embodiment, the information can be recorded on a solid-state material surface with very high density. The information can be used for biometric identification of an individual, covert or overt, for security proposes or for identification of accident victims. For example, the information may include an individual's blood type, allergies and other types of data. The information can be recorded on the lingual surface of a tooth and can easily be read with standard optical methods, such as CCD camera or magnifying optics. In this case, the most effective method of recording is modulation of coefficient of absorption. Carbon nano particles can be used for this purpose. For esthetic reasons, identification information on the labial surface of anterior teeth can be recorded using modulation of refractive index, such as spatial grating, or using fluorescence substance or absorption substance in ultraviolet or infrared wavelength range. In one embodiment, etching of the hard tissue surface can be done through a mask, such as polymer film, with an opening, such as text or a picture. As a result, the text or the picture will form as a porous layer on the hard tissue surface. After this step, absorption or fluorescence nano particles are injected into the porous layer and solidified using polymer coating or via selective heating using one of the methods and apparatuses described above. In another embodiment, laser beam with computer-controlled scanner can be used for recording text or a picture.

Treatment and Repair of Dental Restorative Material

The proposed methods and apparatus for modification of the hard tissue surface can be used to modify and/or repair dental restorative materials, including, but not limited to (a) sealing of crown margins, (b) repairing fractured porcelain intra-orally, and (c) finishing porcelain post adjustment of crowns and filling material

(a) Crowns and inlays, constructed of metals, ceramic resin materials, frequently fail as a result of a break down in the cement which fixes the restoration to the underlying tooth. The proposed method and apparatus can be used to provide a seal to the margin, thereby decreasing post insertion sensitivity due to marginal leakage, marginal breakdown and resulting recurrent caries. In one embodiment, solid-state nano and micro particles are impregnated into the margin, with a fluidity temperature lower or close to the temperature of melting of the restorative material and of the enamel. During selective heating, the melted particles fill the margin, forming a ceramic layer with mechanical, chemical and esthetic properties closely matching those of the restoration. In another embodiment compound preheated in handpiece (FIG. 12) is impregnating into the margin liquid state and after cooling filled margin and prevent leakage.

(b) All cemented porcelain crowns, bridges and inlays cannot be adequately repaired intraorally once the porcelain fractures. Current repair systems rely on air abrasion and/or acid etching of the fractured porcelain and then curing composite resin onto the damaged porcelain to replace the porcelain fractured. Such repairs are not very effective. Alternative methods require the whole restoration to be removed and redone - an expensive and time-consuming process. The proposed methods and apparatus can be used to repair fractures of restorative material intraorally, by impregnation of solid-state nano and micro particles into the fractures, with a fluidity temperature lower than the temperature of melting of the restorative material. During selective heating, the melted particles fill the pores of the restorative material and fuse with it, forming a ceramic layer with mechanical, chemical and esthetic properties closely matching those of the restoration.

(c) The overwhelming majority of laboratory formed ceramic restorations require occlusal adjustments, usually with diamond-coated burs, to correct the occlusion upon insertion of the restoration. This leaves a roughened porcelain surface, which leads to excessive wear of opposing teeth, hastens porcelain fracture and can be uncomfortable to the patient's tongue, lips and cheeks. Ideally, such a surface is reglazed it in a furnace. However, most dentists do not have such furnaces in their practices and are unfamiliar with their use. This necessitates returning the restoration to the laboratory for reglazing, needing another insertion appointment and perhaps another injection for insertion. The proposed method and apparatus can be used for intraoral reglazing of ceramic restorations or other finishing of ceramic surface. The reglazing can be conducted by selective heating and melting of surface of ceramic. In another embodiment over coating on ceramic can be applied using methods and apparatus described above.

### OTHER EMBODIMENTS

While the invention has been described in conjunction with the detailed description thereof, the foregoing description is intended to illustrate and not to limit the scope of the invention, which is defined by the scope of the appended claims. Other aspects, advantages, and modifications are within the scope of the following claims. The use of “such as” and “for example” are only for the purposes of illustration and do not limit the nature or items within the classification.

What is claimed is:

1. A method of modifying dental hard tissue comprising:
forming a superficial microtextured layer on a surface of the dental hard tissue by controlled patterned laser scanning microtexturing of the dental hard layer;
impregnating the superficial microtextured layer with a compound capable of polymerizing when exposed to light to form an impregnated superficial microtextured layer; and
modifying the dental hard tissue by exposing the compound to light to induce polymerization of the compound and its bonding with the impregnated superficial microtextured layer on the surface of the dental hard tissue.

2. The method of claim 1, wherein forming the superficial microtextured layer on the dental hard tissue comprises forming a plurality of microholes or microgrooves having a depth from about 0.5 μm to about 500 μm, a width from about 1 μm to about 250 μm, and a fill factor from about 5% to about 100%.

3. The method of claim 1, wherein the plurality of microholes or microgrooves forms a periodic structure.

4. A method of modification of dental hard tissue comprising:
forming a superficial microtextured layer on a surface of the dental hard tissue by controlled patterned laser scanning microtexturing of the dental hard layer;
impregnating the superficial microtextured layer with a compound and forming an impregnated superficial microtextured layer;
selectively heating the impregnated superficial microtextured layer to a temperature sufficient to fuse the impregnated superficial microtextured layer; and
modifying the dental hard tissue by letting the impregnated superficial microtextured layer to solidify.

5. The method of claim 4, wherein the compound comprises solid or liquid particles.

6. The method of claim 5, wherein the particles are inorganic particles.

7. The method of claim 5, wherein the particles are organic particles.

8. The method of claim 6, wherein the inorganic particles are selected from the group comprising fluoride, germinate, phosphate, lanthanum, zirconium, and silica glasses, porcelain, crystals of quartz, diamond, sapphire, topaz, amethyst, zircon, agate, granite, spinel, fianite, tanzanite, tourmaline crystals selected from the group containing of Ca(NO₃)₂, Ca(OH)₂, BaO₂, CdCl₂, Na₂O—Al₂O₃—SiO₂, Ca(PO₃), CaF₂, Ca₁₀(PO₄)₆(OH)₂, and Ca₁₀(PO₄)₆F₂ and combinations thereof.

9. The method of claim 4, wherein selectively heating the impregnated superficial microtextured layer comprises heating by heat conduction from a heated surface, acoustic energy, electromagnetic energy, comprising light, microwave, radio frequency, and electric current, and combinations thereof.

10. The method of claim 4, wherein forming the superficial microtextured layer on the dental hard tissue comprises forming a plurality of microholes or microgrooves having a depth from about 0.5 μm to about 500 μm, a width from about 1 μm to about 250 μm, and a fill factor from about 5% to about 100%.

11. The method of claim 4, further comprising measuring a temperature of the impregnated superficial microtextured layer during heating.
